(19) United States
(12) Patent Application Publication
(10) Pub. No.: US 2004/0121344 A1
(43) Pub. Date: Jun. 24, 2004

(54) Method of detecting nucleotide polymorphism

(76) Inventors: Yutaka Takarada, Shiga (JP); Toshiya Aono, Shiga (JP); Masaya Segawa, Shiga (JP); Satoko Yoshiga, Shiga (JP)

Correspondence Address: Neil M McCarthy, Kenyon & Kenyon, Suite 700, 1500 K Street NW, Washington, DC, 20005-1257, US

(21) Appl. No.: 10/332,211

(22) PCT Filed: Jul. 5, 2001
(86) PCT No.: PCT/JP01/05792

(30) Foreign Application Priority Data
Jul. 5, 2000 (JP) 2000-203508

(51) Int. Cl. C12Q 1/68; C12P 19/34

(52) U.S. Cl. 435/6; 435/91.2

(57) ABSTRACT

The present invention provides a method for detecting nucleotide polymorphism contained in a sample wherein nuclease activity and legase activity are employed at the same time.

### TECHNICAL FIELD

The present invention relates to a method for analyzing nucleic acid sequences. Specifically, the present invention relates to a nucleic acid sequence analyzing method by which nucleotide polymorphisms contained in specific nucleic acid sequences can be detected. The nucleic acid sequence analyzing method of the present invention is useful in genetic engineering, molecular biology, and related industrial fields.

### BACKGROUND OF THE INVENTION

In the present invention, a nucleotide polymorphism is a genotype having a nucleotide sequence different from that of the wild type. In drug metabolism, polymorphic genes are key factors responsible for variances between individuals in side effects and drug treatment failure. Nucleotide polymorphisms are also known to cause individual variations in the basal metabolism and the like, which are known as constitution. In addition, they serve as genetic labels for various diseases. Consequently, the analysis of such mutation is clinically important, and routine phenotype classification is particularly recommended for clinical studies involving psychiatric patients and suicidal subjects (Gram and Brsen, European Consensus Conference on Pharmacogenetics, Commission of the European Communities, Luxembourg, 1990, pp. 87-96; Balant et al., Eur. J. Clin. Pharmacol. Vol. 36, pp. 551-554 (1989)). For such reasons, there is a demand for methods of analyzing nucleic acid sequences for detecting the various genotypes after identifying the responsible polymorphic genes.

Known techniques for nucleic acid sequence analysis include, for example, the nucleic acid sequencing method (sequencing method). The sequencing method can detect and identify nucleotide polymorphisms contained in nucleic acid sequences, but considerable time and effort are required for template preparation, polymerase reactions, polyacrylamide gel electrophoresis, nucleic acid sequence analysis, etc. Automatic sequencers have simplified this process in recent years; however, the method has a problem of requiring expensive equipment.

As an alternative method, southern hybridization (Masami MURAMATSU Labo. manual gene engineering revised version, MARUZEN CO., LTD. pp. 70-75) can be cited. Employing this method makes it possible to identify a domain of DNA having a nucleotide sequence complementary to that of a labeled DNA probe. In other words, in southern hybridization, a nucleic acid fragment is subjected to electrophoresis on a flat plate of agarose gel or polyacrylamide gel, separated according to the size (length) of the fragment, and denatured into a single chain nucleic acid. Then, a membrane of nitrocellulose, nylon, or the like is attached to the flat plate. The nucleic acid fragment is transferred with its exact electrophoretic pattern intact, and immobilized. Thereafter, a hybrid is formed by the transferred nucleic acid fragment and a nucleotide polymorphism specific DNA probe that is labeled with an RI (a radioactive isotope) or the like, and the nucleic acid fragments on the membrane that are complementary to the probe are detected using autoradiography, etc.

Southern hybridization makes it possible to determine the electrophoretic position and molecular weight of the target nucleic acid fragment. However, the method has certain drawbacks, i.e., prompt analysis is prevented by the time-consuming electrophoresis, autoradiography operation, etc., and, identification of similar sequences for detecting the presence of nucleotide polymorphisms becomes difficult, unless the specificity of the probe is exact, since it performs detection only by determining whether or not the nucleic acid hybridizes with a nucleotide polymorphism specific DNA probe.

In recent years, methods using ligase (Japanese Examined Patent Publication No. 1994-44880 and Patent No. 2622255) and other methods using nuclease (Japanese Unexamined Patent Publication No. 1990-20298 and Japanese Unexamined Patent Publication No.1991-43098) have become known for identifying nucleic acid sequences using enzymes.

An object of the present invention is to provide a nucleic acid sequence analyzing method which can readily detect the type of nucleotide polymorphism of a specific nucleotide-polymorphism-containing nucleic acid sequence in a sample.

### BRIEF DESCRIPTION OF THE DRAWINGS

FIG. 1 shows the positions of the oligonucleotides used in the Examples of the present invention.

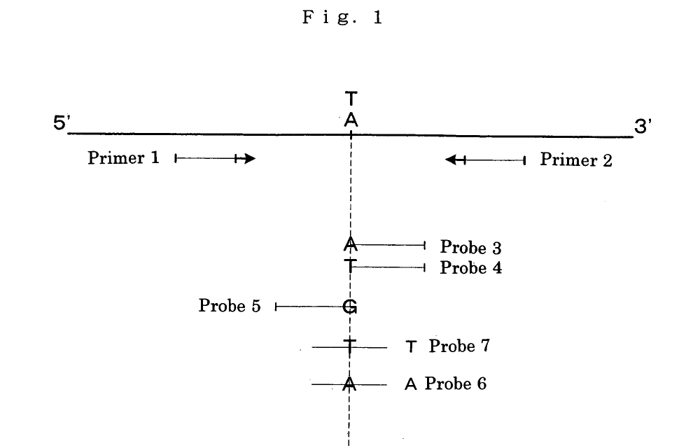

### DISCLOSURE OF THE INVENTION

In view of the above-mentioned problems, the present inventors conducted extensive research and found that nucleotide polymorphisms can be readily detected by using ligase activity and nuclease activity sequentially and/or simultaneously.

Specifically, the inventors found that nucleotide polymorphisms can be detected by hybridizing (1) a first wild-type oligonucleotide having a sequence (bases) complementary to wild-type bases in a nucleotide polymorphism site, and a second oligonucleotide adjacently hybridized with the first wild-type oligonucleotide and having a nucleotide sequence not complementary to the wild-type bases in the nucleotide polymorphism site; and (2) one or more first polymorphism oligonucleotides having a sequence (bases) complementary to polymorphism bases in a polymorphism site, and a second oligonucleotide adjacently hybridized with the first polymorphism oligonucleotide and having a nucleotide sequence not complementary to polymorphism bases in the nucleotide polymorphism site, both (1) and (2) being complementary to one strand of a chromosome or nucleic acid fragment (hereinafter a nucleic acid fragment may be simply referred to as a nucleic acid) having a specific nucleotide polymorphism site; and by detecting nuclease activity and ligase activity using nuclease activity and ligase activity.

More specifically, when only the uncomplementary portion of the nucleotide polymorphism site of the second oligonucleotide is accurately deleted by 5′ exonuclease or 3′ exonuclease, each oligonucleotide can be bound by ligase. The invention is accomplished by the above findings.

Namely, the present invention comprises the following.

Item 1. A method for detecting nucleotide polymorphisms contained in a sample, wherein nuclease activity and ligase activity are used sequentially and/or simultaneously.

Item 2. The method according to Item 1, which detects nucleotide polymorphisms of a nucleotide-polymorphism-containing specific nucleic acid sequence in a sample, wherein an enzyme having nuclease activity and an enzyme having ligase activity are used sequentially and/or simultaneously.

Item 3. The method according to Item 1, wherein the enzyme having nuclease activity and the enzyme having ligase activity are the same.

Item 4. The method according to Item 1, wherein the enzyme having nuclease activity is at least one type of enzyme selected from the group consisting of Mung bean nuclease, S1 nuclease, and exonucleases I-VII.

Item 5. The method according to Item 2, wherein the enzyme having nuclease activity is at least one type of enzyme selected from the group consisting of T4DNA ligase, Ecoli DNA ligase, and RNA ligase.

Item 6. The method according to Item 1, wherein the enzyme having nuclease activity and the enzyme having ligase activity are heat-resistant enzymes.

Item 7. The method according to Item 6, wherein the heat-resistant enzymes are derived from Tth, Taq, KOD, or Pfu.

Item 8. The method according to Item 1, wherein the enzyme having nuclease activity is DNA polymerase.

Item 9. The method according to Item 8, wherein the DNA polymerase is a heat-resistant enzyme derived from Tth, Taq, KOD, or Pfu.

Item 10. A method for identifying a nucleotide polymorphism of a nucleotide-polymorphism-containing chromosome or nucleic acid fragment in a sample, which comprises the steps of:

(1) preparing a first oligonucleotide that has a wild type and/or nucleotide polymorphism sequence site of the chromosome or nucleic acid fragment, and a second oligonucleotide that is complementary to the chromosome or nucleic acid fragment of a strand chain with which the first oligonucleotide is hybridized and that contains a nonhybridized sequence in the nucleotide polymorphism sequence site,

(2) hybridizing the first oligonucleotide and the second oligonucleotide with the chromosome or nucleic acid fragment; and

(3) applying ligase activity, after using nuclease activity to delete the nucleotide sequence that forms no base pairs such as those that are formed in the nucleotide polymorphism sequence site of the second oligonucleotide.

Item 11. The method according to Item 10, wherein a single oligonucleotide obtained by binding each oligonucleotide is detected using a detection probe.

Item 12. The method according to Item 10, wherein the nucleotide polymorphism sequence site has a single base and the base is anticipated to be a nucleotide polymorphism.

Item 13. The method according to Item 10, wherein bases that form no base pairs in the nucleotide polymorphism sequence site that is formed when each oligonucleotide is hybridized are deleted by nuclease activity, then each oligonucleotide is formed into a single oligonucleotide by applying ligase activity, and these processes are repeatedly conducted.

Item 14 . The method according to Item 10, wherein at least one of the first oligonucleotide and the second oligonucleotide is labeled in advance.

Item 15. The method according to Item 14, wherein the labeling is conducted using at least one member selected from the group consisting of enzyme, biotin, fluorescent material, hapten, antigen, antibody, radioactive material, luminophore, and specific nucleic acid sequences.

Item 16. The method according to Item 11, which comprises a second oligonucleotide, wherein the sequence site that forms no base pairs in the nucleotide polymorphism sequence site of the second oligonucleotide and the first oligonucleotide hybridized with a target sequence are bound to different labels, and detection of whether or not the sequences are deleted can be conducted based on the two types of labels when the sequence that forms no base pairs is deleted by nuclease activity.

Item 17. The method according to Item 1, wherein the nucleotide polymorphism contains at least one of a single nucleotide polymorphism, an inserted polymorphism, and a deleted polymorphism.

Item 18. A method for identifying a nucleotide polymorphism of a nucleotide-polymorphism-containing chromosome or nucleic acid fragment in a sample, which comprises the steps of:

(1) preparing a first oligonucleotide that has a nucleotide polymorphism sequence site of the chromosome or nucleic acid fragment at the 3′ end, and a second oligonucleotide that has a sequence position adjacent to the 3′ side of the first oligonucleotide and hybridizes with the chromosome or nucleic acid fragment when the first oligonucleotide is hybridized with the chromosome or nucleic acid fragment, and that contains at least one base on the 5′ end that does not hybridize with the chromosome or nucleic acid fragment,

(2) hybridizing the first oligonucleotide and the second oligonucleotide with a specific nucleic acid, and

(3) binding each oligonucleotide by applying ligase activity, after using nuclease activity to delete the nucleotide sequence site that forms no base pairs, which is formed in the second oligonucleotide at the nucleotide polymorphism sequence when each oligonucleotide is hybridized.

Item 19. The method for identifying a nucleotide polymorphism of a nucleotide-polymorphism-containing chromosome or nucleic acid fragment in a sample according to Item 18, wherein an enzyme having nuclease activity and an enzyme having ligase activity are used sequentially and/or simultaneously.

Item 20 . The method according to Item 18, wherein the enzyme having nuclease activity and the enzyme having ligase activity are the same.

Item 21. The method according to Item 18, wherein the enzyme having nuclease activity is at least one type of enzyme selected from the group consisting of Mung bean nuclease, S1 nuclease, and exonucleases I-VII.

Item 22. The method according to Item 18, wherein the enzyme having ligase activity is at least one type of enzyme selected from the group consisting of T4DNA ligase, Ecoli DNA ligase, and RNA ligase.

Item 23. The method according to Item 18, wherein the enzyme having nuclease activity and the enzyme having ligase activity are heat-resistant enzymes.

Item 24. The method according to Item 23, wherein the heat-resistant enzymes are derived from Tth, Taq, KOD, or Pfu.

Item 25. The method according to Item 18, wherein the enzyme having nuclease activity is DNA polymerase.

Item 26. The method according to Item 25, wherein the DNA polymerase is a heat-resistant enzyme derived from Tth, Taq, KOD, or Pfu.

Item 27. A method for identifying the nucleotide polymorphism of a nucleotide-polymorphism-containing chromosome or nucleic acid fragment in a sample, which comprises the steps of:

(1) preparing a first oligonucleotide that has a nucleotide polymorphism sequence site of the chromosome or nucleic acid fragment at the 5′ end, and a second oligonucleotide, when the first oligonucleotide is hybridized with the chromosome or nucleic acid fragment, that has a sequence position adjacent to the 5′ side of the first oligonucleotide and hybridizes with the chromosome or nucleic acid fragment and that contains at least one base on the 3′ end that does not hybridize with the chromosome or nucleic acid fragment,

(2) hybridizing the first oligonucleotide and the second oligonucleotide with a specific nucleic acid, and

(3) binding each oligonucleotide by applying ligase activity, after using nuclease activity to delete the nucleotide sequences that form no base pairs such as those that are formed in the second oligonucleotide at the nucleotide polymorphism sequence site when each oligonucleotide is hybridized.

Item 28. The method for identifying a nucleotide polymorphism of a nucleotide-polymorphism-containing chromosome or nucleic acid fragment in a sample according to Item 27, wherein an enzyme having nuclease activity and an enzyme having ligase activity are used sequentially and/or simultaneously.

Item 29. The method according to Item 27, wherein the enzyme having nuclease activity and the enzyme having ligase activity are the same.

Item 30. The method according to Item 27, wherein the enzyme having nuclease activity is at least one type of enzyme selected from the group consisting of Mung bean nuclease, S1 nuclease, and exonucleases I-VII.

Item 31. The method according to Item 27, wherein the enzyme having ligase activity is at least one type of enzyme selected from the group consisting of T4DNA ligase, Ecoli DNA ligase, and RNA ligase.

Item 32. The method according to Item 27, wherein the enzyme having nuclease activity and the enzyme having ligase activity are heat-resistant enzymes.

Item 33. The method according to Item 32, wherein the heat-resistant enzyme is derived from Tth, Taq, KOD, or Pfu.

Item 34. The method according to Item 27, wherein the enzyme having nuclease activity is DNA polymerase.

Item 35. The method according to Item 34, wherein the DNA polymerase is a heat-resistant enzyme derived from Tth, Taq, KOD, or Pfu.

Item 36. A kit for identifying the nucleotide polymorphism of a nucleotide-polymorphism-containing chromosome or nucleic acid fragment in a sample, wherein the kit comprises (i) at least one type of first oligonucleotide selected from a group consisting of a wild-type first oligonucleotide and one or two types of polymorphism first oligonucleotide, (ii) a second oligonucleotide that is complementary to the chromosome or nucleic acid fragment of a strand chain with which the first oligonucleotide is hybridized and that contains a nonhybridized sequence in the nucleotide polymorphism sequence site, (iii) at least one type of enzyme having nuclease activity and ligase activity, and (iv) a detection probe, wherein the detection probe can detect hybridized products of first oligonucleotides and second oligonucleotides obtained by applying ligase activity, after using nuclease activity to delete the nucleotide sequences that do not form base pairs in the nucleotide polymorphism sequence site such as those that are formed in the polymorphism sequence site when each oligonucleotide is hybridized.

Item 37. The method according to Item 18, wherein the nucleotide polymorphism contains at least one member selected from the group consisting of a single nucleotide polymorphism, an inserted polymorphism, and a deleted sequence polymorphism.

The present invention is described in more detail below. There are no particular limitations on the chromosome or fragment thereof having a specific polymorphism site that is contained in a sample, as long as it is a target nucleic acid containing a nucleotide polymorphism site that carries the objective genetic information. Examples of such target nucleic acids include Alu sequences, exons and introns that are genes encoding proteins, promoters, etc. More specific examples include genetic materials that affect various diseases, including hereditary diseases, drug metabolism, and lifestyle disorders (high blood pressure, diabetes, etc.). Examples include the ACE gene in the case of high blood pressure.

In the present invention, a polymorphism nucleic acid of a chromosome or nucleic acid fragment includes a wild-type nucleic acid having at least one nucleotide replaced by another nucleotide due to point mutation, and a nucleic acid containing an inserted or deleted sequence in part of the wild-type nucleic acid. It is known that such nucleotide polymorphisms affect the constitution and the like. The method of the present invention is designed to investigate whether or not nucleic acids in a sample have such anticipated polymorphisms.

The method for detecting nucleotide polymorphisms of the present invention is characterized in that nuclease activity and ligase activity are used sequentially and/or simultaneously. The activities can be applied by enzymes, and it is also possible to use an enzyme having both activities.

Specifically, examples of enzymes having nuclease activity include Mung bean nuclease, S1 nuclease, exonucleases I-VII, etc., and examples of enzymes having ligase activity include T4DNA ligase, E.coli DNA ligase, RNA ligase, etc.

It is preferable that the enzymes having nuclease activity and enzymes having ligase activity be heat-resistant enzymes. Preferably, the heat-resistant enzymes are derived from Tth, Taq, KOD, or Pfu.

It is also preferable that the enzyme having nuclease activity be DNA polymerase. More specifically, it is preferable that the DNA polymerase be a heat-resistant enzyme derived from Tth, Taq, KOD, or Pfu.

The specific mode of the method for detecting nucleotide polymorphisms of the present invention is such that the method identifying the nucleotide polymorphisms of a nucleotide-polymorphism-containing chromosome or nucleic acid fragment in a sample comprises the steps of:

(1) preparing a first oligonucleotide having the nucleotide polymorphism sequence site of the chromosome or nucleic acid fragment, and a second oligonucleotide that is complementary to the chromosome or nucleic acid fragment of a strand chain with which the first oligonucleotide is hybridized and that has sequences to which the nucleotide polymorphism sequence site overlaps;

(2) hybridizing the first oligonucleotide and the second oligonucleotide with the chromosome or nucleic acid fragment; and

(3) applying ligase activity, after using nuclease activity to delete the bases that form no base pairs in the nucleotide polymorphism sequence site such as those that are formed when each oligonucleotide is hybridized,

for identifying the nucleotide polymorphism of the specific nucleic acid sequence contained in the sample.

Specifically, the first oligonucleotide is an oligonucleotide having bases complementary to the site of the sequence where the nucleotide polymorphism is anticipated. Preferably, the site where the nucleotide polymorphism is anticipated is the 3′ or 5′ end of the first nucleotide.

When the first oligonucleotide is closer to the 5′ end than the second oligonucleotide, the nucleotide polymorphism site is on the 3′ end of the first oligonucleotide. When the first oligonucleotide is closer to the 3′ end than the second oligonucleotide, the nucleotide polymorphism site is on the 5′ end of the first nucleotide, and the first nucleotide further comprises a phosphoric acid group at 5′.

The second oligonucleotide is an oligonucleotide having bases not complementary to the sequence site where the nucleotide polymorphism is anticipated. The second oligonucleotide overlaps with the first oligonucleotide on at least one of the bases.

The length of each oligonucleotide in the present invention is 13-35 bases, and preferably 16-30 bases.

For example, when the 3′ end base of the first oligonucleotide is designed to be an oligonucleotide where a polymorphism site is anticipated, in the case of a wild-type first, oligonucleotide, a base complementary to the above-described base that is a wild-type nucleic acid is arranged, and, in the case of a polymorphism first oligonucleotide, a base complementary to the above-described base that is a polymorphism nucleic acid is arranged. Furthermore, when the 5′ end of the second oligonucleotide is designed to overlap with the first oligonucleotide in one base, the overlapping base of the second oligonucleotide should be selected from the non-complementary bases both in the wild-type case and polymorphism case. By selecting a non-complementary base in both the wild type and polymorphism cases, it becomes sufficient to prepare only one kind of second oligonucleotide. The examples of possible combinations are as follows:

TABLE 1

| Wild-type | A | | | G | | | C | | | T | | |
|---|---|---|---|---|---|---|---|---|---|---|---|---|
| nucleic acid | | | | | | | | | | | | |
| 3′ end of | T | | | C | | | G | | | A | | |
| wild-type first | | | | | | | | | | | | |
| oligonucleotide | | | | | | | | | | | | |
| Nucleic acid of | G | C | T | A | T | C | A | T | G | G | C | A |
| polymorphism | | | | | | | | | | | | |
| 3′ end of | C | G | A | T | A | G | T | A | C | C | G | T |
| polymorphism | | | | | | | | | | | | |
| first | | | | | | | | | | | | |
| oligonucleotide | | | | | | | | | | | | |
| 5′ end of | A | A | G | G | G | A | C | C | A | T | T | G |
| second | G | C | C | A | T | T | A | T | T | G | C | C |
| oligonucleotide | | | | | | | | | | | | |

When such a design is employed according to the above-described principle, only in the combinations of wild-type first oligonucleotide/second oligonucleotide/wild-type nucleic acid and polymorphism first nucleotide/second oligonucleotide/polymorphism nucleic acid, the first oligonucleotide and the second oligonucleotide are bound into a single chain, since they completely correspond to each other except in the overlapped site of the second oligonucleotide. However, in the combinations of wild-type first oligonucleotide/second oligonucleotide/polymorphism nucleic acid and polymorphism first nucleotide/second oligonucleotide/wild-type nucleic acid, binding by ligase activity does not occur, since the 3′ end of the first oligonucleotide is not complementary, the nuclease activity does not function, and the overlapped site of the second oligonucleotide is not deleted.

Nuclease Activity and Ligase Activity

In the present invention, the wild-type first oligonucleotide and the polymorphism first oligonucleotide are applied to a sample separately or simultaneously.

i) The aforementioned first oligonucleotide and second oligonucleotide are hybridized with a chromosome or nucleic acid fragment containing a nucleotide polymorphism. The hybridization conditions can be those generally employed. For example, hybridization can be performed following the steps described in Molecular Cloning.

ii) The overlapped site of the second oligonucleotide is cut using nuclease activity. Examples of usable enzymatic activities are as described above. Among those, exonuclease activity is preferable and exonucleases such as DNA polymerase or the like are preferably used.

When the 5′ end of the second oligonucleotide is overlapped with the first oligonucleotide, the overlapped site of the second oligonucleotide is cut using a 5′ exonuclease to make a phosphoric acid group to be revealed.

When the 3′ end of the second oligonucleotide is overlapped with the first oligonucleotide, the overlapped site of the second oligonucleotide is cut using a 3′ exonuclease.

iii) At the same time, an oligonucleotide at the 5′ end and an oligonucleotide at the 3′ end are bound into a single oligonucleotide using ligase activity.

In the present invention, if the site corresponding to the nucleotide polymorphism of the first oligonucleotide is not complementary, even when cut by an endnuclease, it is impossible to bind it into a single oligonucleotide using ligase activity, since the site corresponding to the nucleotide polymorphism is not complementary.

When nuclease activity and ligase activity are applied to a sample nucleic acid using a wild-type first oligonucleotide, a single chain is formed if the sample nucleic acid is of the wild type but no reaction occurs if the sample nucleic acid is a polymorphism. On the other hand, when nuclease activity and ligase activity are applied to a sample nucleic acid using a polymorphism first oligonucleotide, a single chain is formed if the sample nucleic acid is a polymorphism but no reaction occurs if the sample nucleic acid is of the wild type. Consequently, it is possible to accurately determine whether a sample nucleic acid is of the wild type or a polymorphism by splitting a single sample into two batches and investigating whether a reaction occurs using the wild type with one batch and the polymorphism with the other. In particular, humans and other higher organisms inherit two forms of each gene, one from the mother and one from the father, and this method makes it possible to determine whether a sample gene is wild-type homozygous, polymorphism homozygous, or heterozygous. In the case where the sample gene is heterozygous, both the wild-type gene and polymorphism gene are present, so that the reaction occurs both where the wild type is used and polymorphism is used.

By applying different labels to the wild-type first oligonucleotide and the polymorphism first oligonucleotide, it becomes unnecessary to split a single sample into two batches.

If the target nucleic acid is not in sufficient quantity for detection, the nucleic acid fragment containing the aforementioned polymorphism sequence can be amplified by the amplification reaction described below prior to the above-described hybridization.

There is no limitation on the amplification method and it is possible to employ a method such as PCR (Polymerase chain reaction: Japanese Examined Patent Publication No. 1992-67960, Japanese Examined Patent Publication No. 1992-67957), NASBA (Nucleic acid sequence-based amplification method: Nature Vol. 350 p. 91 (1991)), LCR (WO89/12696, Japanese Unexamined Patent Publication No. 1990-2934, etc.), SDA (Strand Displacement Amplification: Nucleic Acid Research Vol. 20 p. 1691 (1992)), RCR (WO90/01069), TMA (Transcription Mediated amplification Method: J. Clin. Microbiol. Vol. 31 p. 3270 (1993)), etc.

Detection

The oligonucleotide of a single chain obtained by the above reaction is detected in the following manner using a probe.

It is preferable that the probes used in the present invention contain nucleotide polymorphisms, and detection signals vary depending on the kind of nucleotide polymorphism. Preferably, the probe should have a nucleotide sequence having at least 15 or more continuous bases and more preferably 18 or more continuous bases. Preferably, the probe has a sequence complementary to both a portion that contains the nucleotide polymorphism site of the first oligonucleotide and a portion of the second oligonucleotide. The probe can be DNA, RNA, or PNA, as long as it forms a chain complementary to a specific nucleic acid sequence, and it can be prepared by chemical synthesis or by a biological method. The probe can be synthesized by the phosphoamidite method using a 391 Perkin-Elmer DNA synthesizer. Purification can be conducted by FPLC using a MONO-Q column or a reversed column. Alternative methods include the phosphoric acid triester method, H-phosphonate method, thiophosphite method, etc.

It is preferable that the first oligonucleotide or the second oligonucleotide be labeled in advance. In this case, a modified base can be introduced by inserting a nucleotide that contains biotin, a linker arm, a fluorescent material, etc., oligo dGTP, oligo dATP, oligo dTTP, oligo dCTP, or the like to the 5′ end or 3′ end. Alternatively, a modified base can be introduced by substituting the nucleotide in the oligonucleotide sequence with a nucleotide containing biotin, a linker arm, a fluorescent material, or the like, and conducting synthesis. It is also possible to introduce known oligonucleotide labels, such as ³²P, ³⁵S, and like radioactive substances; ALP, POD, and like enzymes; FITC, HEX, 6-FAM, TET, and like fluorescent materials, etc., to the synthesized nucleotide.

Specifically, for example, the first oligonucleotide and the second oligonucleotide labeled with biotin or the like are hybridized with specific nucleic acids, dissociated, and hybridized with, for example, a polymorphism-specific probe bound to a solid phase, such as a micro titer plate. Thereafter, the nucleic acids not hybridized are washed out. Detection is then conducted to determine whether the nucleic acid is hybridized with the probe according to the label of the first oligonucleotide hybridized with the probe, and the type of polymorphism, such as wild type, variant type, or mixed type, is identified according to the ratio of the detection signal of each probe.

By applying different labels, such as fluorescent labels having different colors, to wild-type and polymorphism oligonucleotides, detection can be performed in wells on a single plate.

Kit

In the present invention, a kit is characterized as containing (i) at least one type of first oligonucleotide selected from a group consisting of a wild-type first oligonucleotide and one or two types of polymorphism first oligonucleotides, (ii) a second oligonucleotide that is complementary to the chromosome or nucleic acid fragment of a strand chain with which the first oligonucleotide is hybridized and that contains sequences not hybridized in the nucleotide polymorphism sequence site, (iii) at least one enzyme having nuclease activity and ligase activity, and (iv) a detection probe that can detect hybridized products of first oligonucleotides and second oligonucleotides obtained by applying ligase activity, after using nuclease activity to delete the nucleotide sequences that form no base pairs such as those that are formed in the nucleotide polymorphism sequence site when each oligonucleotide is hybridized.

The first oligonucleotide and the second oligonucleotide may be labeled in advance by the aforementioned enzyme, biotin, fluorescent material, hapten, antigen, antibody, radioactive material, or luminophore.

### BEST MODE FOR CARRYING OUT THE INVENTION

The present invention is described in more detail below based on examples. However, the present invention is not limited by the following examples.

### EXAMPLE 1

Detection of Nucleotide Polymorphism in the ACE Gene

(1) Synthesis of Primers for Amplifying the ACE Gene Fragment and Oligonucleotide for Detecting Polymorphism

Oligonucleotides (primers 1, 2) having the nucleotide sequences shown by Seq. Nos. 1 and 2 that have the same sequence as that of the human ACE gene, and oligonucleotides (detection probes 3, 4, 5) having the nucleotide sequences shown by Seq. Nos. 3, 4, and 5 were synthesized by the phosphoamidite method using a 392 Perkin-Elmer DNA synthesizer. The detection probes 3 and 4 have sequences complementary to that of the human ACE gene. However, each of them has a wild-type or polymorphism base corresponding to the nucleotide polymorphism site at the 3′ end, and biotin is connected to the 5′ side thereof. The detection probe 5 has a sequence complementary to that of the human ACE gene adjacent to the 3′ end of the detection probes 3 and 4; however, the single base at its 5′ end corresponds to the nucleotide polymorphism site and is not complementary to the wild type or the polymorphism. Synthesis was performed according to the manual, and the various oligonucleotides were deprotected overnight at 55° C. with ammonia water. Purification of the oligonucleotides was conducted using a Perkin-Elmer OPC column.

(2) Synthesis of Oligonucleotide Having a Linker Arm

An oligonucleotide having the nucleotide sequence shown by Seq. No. 6 (A probe) and an oligonucleotide having the nucleotide sequence shown by Seq. No. 7 (T probe) were synthesized by the phosphoamidite method using a 392 Perkin-Elmer DNA synthesizer. Both the A probe and T probe are complementary to the sequence of the human ACE gene and specific in the nucleotide polymorphism sites thereof.

During the synthesizing process, according to the synthesizing method disclosed in Japanese Unexamined Patent Publication No. 1985-500717, uridine having a linker arm at its 5-position prepared by chemically synthesizing deoxyuridine was introduced into the aforementioned oligonucleotide. The synthesized linker oligonucleotides were deprotected overnight with ammonia water at 50° C. and purified using a Perkin-Elmer OPC column.

(3) Binding of Probe Oligonucleotide to Microtiter Plate

The probe oligonucleotide synthesized in item (2) described above was bound to the inner surface of a microtiter plate through the linker arm thereof. The oligonucleotide was diluted with a solution of 50 mM of boric acid buffer solution (pH 10) and 100 mM of MgCl₂ in a manner such that the concentration became 0.05 pmol/μl, and 100 μl of the resultant solution was dispersed into each well of the microtiter plate (MicroFLUOR B, Dynatech Corp) and allowed to stand for about 15 hours at room temperature, binding the linker oligonucleotide to the inner surfaces of the microtiter plate. These were then substituted with 0.1 pmol of dNTP, 0.5% of PVP (polyvinyl pyrolidone), and 5×SSC, then blocked for about 2 hours at room temperature to suppress non-specific reactions, and finally washed in 1×SSC and dried.

(4) Amplification of the Human ACE Gene Fragment by PCR

Using DNA solutions extracted from human keukocytes as samples, human ALDH2 gene fragments were amplified by adding the following reagents under the conditions described below.

Reagents: A 25-μl solution containing the following reagents was prepared.

| | | |
|---|---|---|
| Primer 1 | 10 | pmol |
| Primer 2 | 10 | pmol |
| X 10 buffer | 2.5 | μl |
| 2 mM dNTP | 2.5 | μl |
| Tth DNA polymerase | 1 | U |
| Extracted DNA solution | 100 | ng |

Amplification Conditions

94° C. for 2 minutes

94° C. for 1 minute, 57° C. for 2 minutes, 75° C. for 1.5 minutes (35 cycles)

(5) Detecting Reaction Using Detection Oligonucleotide

Alkaline phosphatase U derived from shrimp was added to 5 μl of the amplification reaction solution described in item (4) and kept at 37° C. for 15 minutes and at 80° C. for 15 minutes. Then, a 20-μl solution containing the following reagents was prepared.

| | | |
|---|---|---|
| Detection oligo 3 or 4 | 10 | pmol |
| Detection oligo 5 | 10 | pmol |
| X 10 buffer | 2 | μl |
| Tth DNA polymerase | 1 | U |
| Tth DNA ligase | 1 | U |
| Amplification reaction solution | 5 | μl |

Detection Conditions

94° C. for 2 minutes

94° C. for 1 minute, 65° C. for 2 minutes (5 cycles)

(6) Hybridization in Microtiter Plates

10 μl of 0.6 N NaOH was added to 10 μl of the detecting reaction solution described in item (5) for denaturing the DNA. Then, the resultant solution was added to 80 μl of solution comprising 200 mM of citric acid-phosphoric acid buffer solution (pH 6.0), 2% SDS (sodium dodecyl sulfate), 750 mM of NaCl, and 0.1% NaN₃ and poured into the micotiter plate described in item (3) to which capture probes were bound. These were covered with liquid paraffin to prevent evaporation, and shaken for 30 minutes at 50° C. In this way, a nucleic acid fragment in which the nuclear oligonucleotide has been hybridized by a detecting reaction, partly deleted by nuclease, and bound by ligase was specifically captured on the microtiter plate by the fixed probes.

Then, these were substituted with 2×SSC (pH 7.0), 1% SDS and also covered with liquid paraffin to prevent evaporation, followed by shaking for 20 minutes at 55° C. Next, these were substituted with 100 μl of a solution obtained by diluting streptoavidin (DAKO: D0396) labeled with alkaline phosphatase by 2,000 times with a solution of 50 mM of tris-hydrochloric acid buffer (pH 7.5) and 1% BSA solution, and shaken for 15 minutes at 37° C. This served to bind the alkaline phosphatase-labeled streptoavidin specifically to the biotin of the captured DNA. After washing this three times in 250 μl of a solution containing 50 mM of tris-hydrochloric acid buffer (pH 7.5) and 0.025% Tween 20 solution, 50 μl of dioxetan compound (Trade name Lumiphos 480, Lumigen Co.), which is the luminescent base of alkaline phosphatase, was added thereto. After keeping it at 37° C. for 15 minutes, luminescence was measured in a dark room using a photon counter (Hamamatsu Photonics KK).

All of these steps were performed automatically with an automated DNA probe measurement system (see the Nihon Rinshokensa Jidokagakukai Kaishi (Journal of the Japanese Association for Automation of Clinical Testing) Vol. 20, Page 728 (1995)), and took about 2.5 hours.

(7) Results of the Measurement and Examination of the Human ACE Gene Polymorphism

Table 1 shows the detection results of human ACE gene polymorphisms that were reacted in the above-described Item (5) and detected in Item (6). The values indicate luminousity (cps: count/second). The A signal represents the detection signal of an amplified nucleic acid fragment that was reacted with an A probe. The T signal represents the detection signal of an amplified nucleic acid fragment that was reacted with a T probe.

TABLE 2

| | Sample type | T signal | A signal |
|---|---|---|---|
| Detection | T/T | 22650 | 170 |
| oligos 3, 5 | T/A | 17250 | 12620 |
| | A/A | 260 | 80 |
| Detection | T/T | 110 | 450 |
| oligos 4, 5 | T/A | 17880 | 11690 |
| | A/A | 120 | 13230 |

As shown in Table 1, the single nucleotide polymorphism of the ACE gene can be identified by the signals obtained by each probe.

The present invention provides a method for accurately and readily detecting a polymorphism in a sample nucleic acid. The method of the present invention is free from false positives and enables readable identification between homozygosis and heterozygosis, which was difficult using the known methods.

1. A method for detecting nucleotide polymorphisms contained in a sample, wherein nuclease activity and ligase activity are used sequentially and/or simultaneously.

2. The method according to claim 1, which detects nucleotide polymorphisms of a nucleotide-polymorphism-containing specific nucleic acid sequence in a sample, wherein an enzyme having nuclease activity and an enzyme having ligase activity are used sequentially and/or simultaneously.

3. The method according to claim 1, wherein the enzyme having nuclease activity and the enzyme having ligase activity are the same.

4. The method according to claim 1, wherein the enzyme having nuclease activity is at least one type of enzyme selected from the group consisting of Mung bean nuclease, S1 nuclease, and exonucleases I-VII.

5. The method according to claim 2, wherein the enzyme having nuclease activity is at least one type of enzyme selected from the group consisting of T4DNA ligase, Ecoli DNA ligase, and RNA ligase.

6. The method according to claim 1, wherein the enzyme having nuclease activity and the enzyme having ligase activity are heat-resistant enzymes.

7. The method according to claim 6, wherein the heat-resistant enzymes are derived from Tth, Taq, KOD, or Pfu.

8. The method according to claim 1, wherein the enzyme having nuclease activity is DNA polymerase.

9. The method according to claim 8, wherein the DNA polymerase is a heat-resistant enzyme derived from Tth, Taq, KOD, or Pfu.

10. A method for identifying a nucleotide polymorphism of a nucleotide-polymorphism-containing chromosome or nucleic acid fragment in a sample, which comprises the steps of:
(1) preparing a first oligonucleotide that has a wild type and/or nucleotide polymorphism sequence site of the chromosome or nucleic acid fragment, and a second oligonucleotide that is complementary to the chromosome or nucleic acid fragment of a strand chain with which the first oligonucleotide is hybridized and that contains a nonhybridized sequence in the nucleotide polymorphism sequence site,
(2) hybridizing the first oligonucleotide and the second oligonucleotide with the chromosome or nucleic acid fragment; and
(3) applying ligase activity, after using nuclease activity to delete the nucleotide sequence that forms no base pairs such as those that are formed in the nucleotide polymorphism sequence site of the second oligonucleotide.

11. The method according to claim 10, wherein a single oligonucleotide obtained by binding each oligonucleotide is detected using a detection probe.

12. The method according to claim 10, wherein the nucleotide polymorphism sequence site has a single base and the base is anticipated to be a nucleotide polymorphism.

13. The method according to claim 10, wherein bases that form no base pairs in the nucleotide polymorphism sequence site that is formed when each oligonucleotide is hybridized are deleted by nuclease activity, then each oligonucleotide is formed into a single oligonucleotide by applying ligase activity, and these processes are repeatedly conducted.

14. The method according to claim 10, wherein at least one of the first oligonucleotide and the second oligonucleotide is labeled in advance.

15. The method according to claim 14, wherein the labeling is conducted using at least one member selected from the group consisting of enzyme, biotin, fluorescent material, hapten, antigen, antibody, radioactive material, luminophore, and specific nucleic acid sequences.

16. The method according to claim 11, which comprises a second oligonucleotide, wherein the sequence site that forms no base pairs in the nucleotide polymorphism sequence site of the second oligonucleotide and the first oligonucleotide hybridized with a target sequence are bound to different labels, and detection of whether or not the sequences are deleted can be conducted based on the two types of labels when the sequence that forms no base pairs is deleted by nuclease activity.

17. The method according to claim 1, wherein the nucleotide polymorphism contains at least one of a single nucleotide polymorphism, an inserted polymorphism, and a deleted polymorphism.

18. A method for identifying a nucleotide polymorphism of a nucleotide-polymorphism-containing chromosome or nucleic acid fragment in a sample, which comprises the steps of:
(1) preparing a first oligonucleotide that has a nucleotide polymorphism sequence site of the chromosome or nucleic acid fragment at the 3′ end, and a second oligonucleotide that has a sequence position adjacent to the 3′ side of the first oligonucleotide and hybridizes with the chromosome or nucleic acid fragment when the first oligonucleotide is hybridized with the chromosome or nucleic acid fragment, and that contains at least one base on the 5′ end that does not hybridize with the chromosome or nucleic acid fragment,
(2) hybridizing the first oligonucleotide and the second oligonucleotide with a specific nucleic acid, and
(3) binding each oligonucleotide by applying ligase activity, after using nuclease activity to delete the nucleotide sequence site that forms no base pairs, which is formed in the second oligonucleotide at the nucleotide polymorphism sequence when each oligonucleotide is hybridized.

19. The method for identifying a nucleotide polymorphism of a nucleotide-polymorphism-containing chromosome or nucleic acid fragment in a sample according to claim 18, wherein an enzyme having nuclease activity and an enzyme having ligase activity are used sequentially and/or simultaneously.

20. The method according to claim 18, wherein the enzyme having nuclease activity and the enzyme having ligase activity are the same.

21. The method according to claim 18, wherein the enzyme having nuclease activity is at least one type of enzyme selected from the group consisting of Mung bean nuclease, S1 nuclease, and exonucleases I-VII.

22. The method according to claim 18, wherein the enzyme having ligase activity is at least one type of enzyme selected from the group consisting of T4DNA ligase, Ecoli DNA ligase, and RNA ligase.

23. The method according to claim 18, wherein the enzyme having nuclease activity and the enzyme having ligase activity are heat-resistant enzymes.

24. The method according to claim 23, wherein the heat-resistant enzymes are derived from Tth, Taq, KOD, or Pfu.

25. The method according to claim 18, wherein the enzyme having nuclease activity is DNA polymerase.

26. The method according to claim 25, wherein the DNA polymerase is a heat-resistant enzyme derived from Tth, Taq, KOD, or Pfu.

27. A method for identifying the nucleotide polymorphism of a nucleotide-polymorphism-containing chromosome or nucleic acid fragment in a sample, which comprises the steps of:
(1) preparing a first oligonucleotide that has a nucleotide polymorphism sequence site of the chromosome or nucleic acid fragment at the 5′ end, and a second oligonucleotide, when the first oligonucleotide is hybridized with the chromosome or nucleic acid fragment, that has a sequence position adjacent to the 5′ side of the first oligonucleotide and hybridizes with the chromosome or nucleic acid fragment and that contains at least one base on the 3′ end that does not hybridize with the chromosome or nucleic acid fragment,
(2) hybridizing the first oligonucleotide and the second oligonucleotide with a specific nucleic acid, and
(3) binding each oligonucleotide by applying ligase activity, after using nuclease activity to delete the nucleotide sequences that form no base pairs such as those that are formed in the second oligonucleotide at the nucleotide polymorphism sequence site when each oligonucleotide is hybridized.

28. The method for identifying a nucleotide polymorphism of a nucleotide-polymorphism-containing chromosome or nucleic acid fragment in a sample according to claim 27, wherein an enzyme having nuclease activity and an enzyme having ligase activity are used sequentially and/or simultaneously.

29. The method according to claim 27, wherein the enzyme having nuclease activity and the enzyme having ligase activity are the same.

30. The method according to claim 27, wherein the enzyme having nuclease activity is at least one type of enzyme selected from the group consisting of Mung bean nuclease, S1 nuclease, and exonucleases I-VII.

31. The method according to claim 27, wherein the enzyme having ligase activity is at least one type of enzyme selected from the group consisting of T4DNA ligase, Ecoli DNA ligase, and RNA ligase.

32. The method according to claim 27, wherein the enzyme having nuclease activity and the enzyme having ligase activity are heat-resistant enzymes.

33. The method according to claim 32, wherein the heat-resistant enzyme is derived from Tth, Taq, KOD, or Pfu.

34. The method according to claim 27, wherein the enzyme having nuclease activity is DNA polymerase.

35. The method according to claim 34, wherein the DNA polymerase is a heat-resistant enzyme derived from Tth, Taq, KOD, or Pfu.

36. A kit for identifying the nucleotide polymorphism of a nucleotide-polymorphism-containing chromosome or nucleic acid fragment in a sample, wherein the kit comprises (i) at least one type of first oligonucleotide selected from a group consisting of a wild-type first oligonucleotide and one or two types of polymorphism first oligonucleotide, (ii) a second oligonucleotide that is complementary to the chromosome or nucleic acid fragment of a strand chain with which the first oligonucleotide is hybridized and that contains a nonhybridized sequence in the nucleotide polymorphism sequence site, (iii) at least one type of enzyme having nuclease activity and ligase activity, and (iv) a detection probe, wherein the detection probe can detect hybridized products of first oligonucleotides and second oligonucleotides obtained by applying ligase activity, after using nuclease activity to delete the nucleotide sequences that do not form base pairs in the nucleotide polymorphism sequence site such as those that are formed in the polymorphism sequence site when each oligonucleotide is hybridized.

37. The method according to claim 18, wherein the nucleotide polymorphism contains at least one member selected from the group consisting of a single nucleotide polymorphism, an inserted polymorphism, and a deleted sequence polymorphism.


SEQUENCE LISTING (7 sequences)

SEQ ID NO: 1 - DNA, 20 bp, Artificial Sequence
  primer
tcgggctgggaagatcgagc

SEQ ID NO: 2 - DNA, 20 bp, Artificial Sequence
  primer
ctctgccccttctcctgcgc

SEQ ID NO: 3 - DNA, 24 bp, Artificial Sequence
  oligonucleotide
tggagaaagggcctcctctcttta

SEQ ID NO: 4 - DNA, 24 bp, Artificial Sequence
  oligonucleotide
tggagaaagggcctcctctctttt

SEQ ID NO: 5 - DNA, 19 bp, Artificial Sequence
  oligonucleotide
ggaagatggggacccggca

SEQ ID NO: 6 - DNA, 20 bp, Artificial Sequence
  probe
cccatcttcaaaagagagga

SEQ ID NO: 7 - DNA, 20 bp, Artificial Sequence
  probe
cccatcttctaaagagagga